(19) United States
(12) Patent Application Publication
(10) Pub. No.: US 2010/0168061 A1
(43) Pub. Date: Jul. 1, 2010

(54) METHOD FOR PREPARING ENZYMATICALLY HIGHLY BRANCHED-AMYLOSE AND AMYLOPECTIN CLUSTER

(75) Inventors: Jin Hee Park, Gyeonggi-do (KR); Sang Hoon Song, Incheon (KR); Kang Pyo Lee, Seoul (KR)

Correspondence Address: LAW OFFICES OF KHALILIAN SIRA, LLC, 9100 PERSIMMON TREE ROAD, POTOMAC, MD, 20854, US

(73) Assignee: CJ CHEILJEDANG CORP, Seoul (KR)

(21) Appl. No.: 12/524,182

(22) PCT Filed: May 9, 2007
(86) PCT No.: PCT/KR2007/002265

§ 371 Date: Jan. 15, 2010

(30) Foreign Application Priority Data
Feb. 1, 2007 (KR) 10-2007-0010737

(51) Int. Cl. A61K 31/718; C12P 19/18; A61P 3/04; A61P 3/10

(52) U.S. Cl. 514/60; 435/97

(57) ABSTRACT

The present invention relates to a method for preparing enzymatically highly branched-amylose and amylopectin cluster. Alpha-glucanotransferase or branching enzyme hydrolyzes the linkage of the segment between amylopectin clusters in starch, producing amylopectin cluster, and simultaneously branching enzyme attaches the branched side-chain to amylose, producing branched amylose, and subsequently treating the amylopectin cluster or branched amylose with maltogenic amylase for cleaving long side chain into short side chain and for transferring glucose to the side chain, generating highly branched amylopectin cluster, highly branched amylose or branched oligosaccharide from starch effectively.

### TECHNICAL FIELD

The present invention relates to a method for preparing enzymatically highly branched amylose and amylopectin cluster, more particularly, to a method which comprises the steps of: hydrolyzing amylopectin with branching enzyme or α-glucanotransferase to produce amylopectin cluster and simultaneously treating amylose with branching enzyme to prepare branched amylose in which the glycosidic side-chain is branched; and preparing highly branched amylopectin cluster, highly branched amylose or branched oligosaccharide from the prepared amylopectin cluster and branched amylose using transglycosylation activity of maltogenic amylase.

### BACKGROUND ART

A method is required for effectively preparing novel functional materials, highly branched amylopectin cluster and amylose, which are high water-soluble and delay its digestion in the small intestine and prevent a surge in the blood glucose level and enhance a power of locomotion by a continuous supply of calorie.

Alpha-glucanotransferase or branching enzyme hydrolyzes the segment between amylopectin clusters, producing amylopectin cluster. Furthermore, branching enzyme generates branched amylose when reacted with amylose. Maltogenic amylase hydrolyzes starch mainly to maltose unit, and it also exhibits high transglycosylation activity via the formation of various glycosidic linkage, which produce highly branched amylopectin and amylose in which glucose is linked at the nonreducing ends by α-1,6 linkage.

### DISCLOSURE

### Technical Problem

Accordingly, it is an object of the present invention to provide novel functional materials, highly branched amylopectin and amylose, using maltogenic amylase, and α-glucanotransferase or branching enzyme.

To attain the above object, amylopectin or amylose was treated with branching enzyme isolated from Bacillus subtilis 168 or α-glucanotransferase isolated from Thermus scotoductus at pH 5.5˜7.5 and 30˜75° C. and subsequently treated with maltogenic amylase isolated from Bacillus stearothermophilus at 45˜65° C., producing highly branched amylopectin cluster and amylose as a result.

### Technical Solution

The present invention is characterized in providing with a method for preparing highly branched amylose, highly branched amylose or branched oligosaccharide by reacting starch with branching enzyme or α-glucanotransferase and maltogenic amylase.

The starch may be selected from starch, starch-containing grains or amylopectin and amylose.

The method of the present invention is characterized in comprising steps of: adding branching enzyme isolated from Bacillus subtilis or α-glucanotransferase isolated from Thermus scotoductus to starch, and incubating at 30˜75° C. for 30 min to 4 hr, generating amylopectin cluster or branched amylose; treating the obtained amylopectin cluster or branched amylose with maltogenic amylase of Bacillus stearothermophilus at 45˜65° C. for 2˜4 hr.

The α-glucanotransferase, branching enzyme and maltogenic amylase may be typical enzymes isolated and purified from natural type of prokaryotic or eukaryotic organism, or may be prepared by an artificial expression of its genes using an recombinant DNA technology.

According to the present invention, the α-glucanotransferase was prepared by means that a gene encoding a α-glucanotransferase of Thermus scotoductus was cloned and transformed into Bacillus subtilis ISW 1214 and its expressed enzyme was purified. The branching enzyme was produced by means that a gene encoding a branching enzyme of Bacillus subtilis 168 was cloned and transformed into Bacillus subtilis and its expressed enzyme was purified. The maltogenic amylase was also prepared by means that a gene encoding a maltogenic amylase of Bacillus stearothermophilus was cloned and transformed into Bacillus subtilis LK87 and its expressed enzyme was purified.

Among the above enzymes, branching enzyme may be isolated from Bacillus subtilis 168 or prepared using genetic engineering method. According to one example of the present invention, branching enzyme is produced by following steps of:

(a) inserting DNA sequence encoding branching enzyme which is isolated from Bacillus subtilis 168 to prepare a recombinant vector; (b) introducing the recombinant vector into host cell to prepare a transformant; and (c) culturing the transformant to produce amylase.

In the (a) step, a sequence encoding branching enzyme may be inserted into a typical expression vector, i.e. pET-22b(+), but it is desirable to select a suitable vector depending on host cell. In one example of the present invention, p6xHTKNd vector having a cleavage map as shown in FIG. 2 was prepared.

In the (b) step of transformation, the host cell may be prokaryotes, eukaryotes or cells derived from eukaryotes, i.e. E. coli, lactic acid bacteria, yeast, fungi etc., but not limited thereby. The method of transformation may be carried out using a typical method publicly known.

In the (c) step of culturing the transformant, medium may be suitably selected depending on the host cell, and its culture condition may be also changed depending on the host cell. When cultured Escherichia coli MC1061, prepared as one example of the present invention, in LB medium containing kanamycin at 30˜37° C. for 16˜20 hr, a number of branching enzymes may be produced.

Whereas, the method for producing branching enzyme may include a purification step of the amylase recombinant protein from the transformant after the (c) step. The purification step may contain steps of sonicating the transformed cell and carrying out Ni affinity chromatography using the sonicated supernatant.

The above method for producing branching enzyme may have effects of reducing costs by culturing microorganism at lower temperature, and furthermore enhancing its productivity.

The amylase of the present invention may be easily prepared using a typical method publicly known, and that is obvious to a person with an ordinary skill in the art to the invention pertains.

The molecular weights and compositions of highly branched amylopectin cluster and amylose, prepared by the above method, are analyzed using SEC-MALLS (Size Exclusion Chromatography-Multi Angle Lase Light Scattering) and high performance ion exchange chromatography respectively. That is, the molecular weights of highly branched amylopectin and amylose may be determined using SEC-MALL (Size Exclusion Chromatography-Multi Angle Lase Light Scattering), and the compositions of highly branched amylopectin and amylose may be analyzed using high performance ion exchange chromatography, after reacting it with isoamylase for debranching.

According to one example of the present invention, highly branched amylopectin cluster may be produced and determined by following steps of:

incubating gelatinized waxy rice starch with 50˜100 U/g (50 unit per 1 g of waxy rice) of α-glucanotransferase to produce amylopectin cluster;

treating the produced amylopectin cluster with 100˜500 U/g (100˜500 unit per 1 g of amylopectin cluster) of maltogenic amylase at 45˜65° C. for 3˜5 hr to produce highly branched amylopectin cluster.

When determined its molecular weight using SEC-MALLS in each step, the molecular weight of amylopectin cluster was determined as about 10⁵ and that of highly amylopectin cluster as approximately 10⁴, in contrast that of waxy rice starch as about 10⁸.

The amylopectin cluster and highly amylopectin cluster may be treated with 0.2˜1 U/mg of isoamylase (0.2˜1 unit per 1 mg of substrate) at 45˜60° C. for 24˜48 hr for debranching α-1,6 linkage, and their compositions may be identified using High-Performance Ion-Exchange Chromatography.

To confirm the highly branched amylopectin cluster, which has more than 13 DP, it should be treated with β-amylase due to the difficulty in identifying its branched degree. It is estimated that highly amylopectin cluster, which has 10⁴˜10⁵ of molecular weight and 6˜23 DP of length of α-1,6 linkage, was produced as a result.

Whereas, according to one example of the present invention, highly branched amylose is produced by following steps of:

incubating amylose (type III derived from potato, Sigma) with 50˜100 U/mg of branching enzyme (50˜100 unit per 1 mg of substrate) to obtain branched amylose;

treating the branched amylose with 0.2˜1 U/g of maltogenic amylase (0.2˜1 unit per 1 mg of substrate) to produce highly branched amylose.

When the branched amylose and highly branched amylose is treated with 0.2˜1 U/mg of isoamylase for debranching, followed by carrying out High-Performance Ion-Exchange Chromatography, it can be determined that highly branched amylose, which has new 8˜18 DP of α-1,6 linkage as well as α-1,4 linkage, is produced.

In addition, according to one example of the present invention, when analyzed oligosaccharides generated from the production of highly branched amylopectin cluster using a silica-gel K5F thin layer chromatography plate, it is confirmed that long maltooligosaccharides are converted to 2˜5 BDP of branched oligosaccharides.

The term “branched” as used in the present invention is defined as a status that glucoses are linked by α-1,6 linkage as well as α-1,4 linkage. In addition, “DP” is an abbreviation for “degree of polymerization”, which means the number of glucoses. Especially in the present invention, it means the number of glucose debranched by isoamylase, that is, the length of branch chain. In the present invention, “highly branched” is defined as DP is more than 6.

In addition, “BDP” is an abbreviation for “branched degree of polymerization”, and branched oligosaccharides are expressed as BDP without the treatment of isoamylase because it has low molecular weight and its branched degree can be determined by TLC assay.

The present invention is characterized in providing with a processed health care food which has an effect of anti-diabetes, anti-obesity or continuous energy supply.

“effect of continuous energy supply”, stated in the present invention, means that the highly branched amylopectin or amylose may be slowly hydrolyzed, resulting in supplying energy continuously.

As shown in Table 1, when compared the hydrolysis kinetics between amylopectin cluster and highly amylopectin cluster by glucoamylase (which can hydrolyze both α-1,4 and α-1,6 linkages, but power of hydrolysis on α-1,4 linkage is more higher), it is estimated that amylopectin cluster is easily hydrolyzed by glucoamylase due to a high K_{cat} and low Kₘ. If kₘ value is low, its efficiency becomes high, which induces a strong bond with a substrate and produces a lot of products. In contrast, if Kₘ value is high, its efficiency becomes low, which generates a weak bond with a substrate. The higher K_{cat} is, the more molecules exist for transferring a substrate to a product. Accordingly, highly amylopectin cluster is estimated to be slowly hydrolyzed by glucoamylase. That is, highly branched form is more slowly hydrolyzed and thereby, can supply energy continuously.

### ADVANTAGEOUS EFFECTS

The present invention relates to a method for preparing enzymatically highly branched-amylose and amylopectin cluster. Alpha-glucanotransferase or branching enzyme hydrolyzes the linkage of the segment between amylopectin clusters in starch, producing amylopectin cluster, and simultaneously branching enzyme attaches the branched side-chain to amylose, producing branched amylose, and subsequently treating the amylopectin cluster or branched amylose with maltogenic amylase for cleaving long side chain into short side chain and for transferring glucose to the side chain, generating highly branched amylopectin cluster, highly branched amylose or branched oligosaccharide from starch effectively.

### DESCRIPTION OF DRAWINGS

FIG. 1 is a schematic diagram to show the procedure for preparing highly amylopectin cluster and amylose from amylopectin or starch using BSMA (Bacillus stearothermophilus maltogenic amylase) and α-GTase (4-α-glucanotransferase) or branching enzyme.

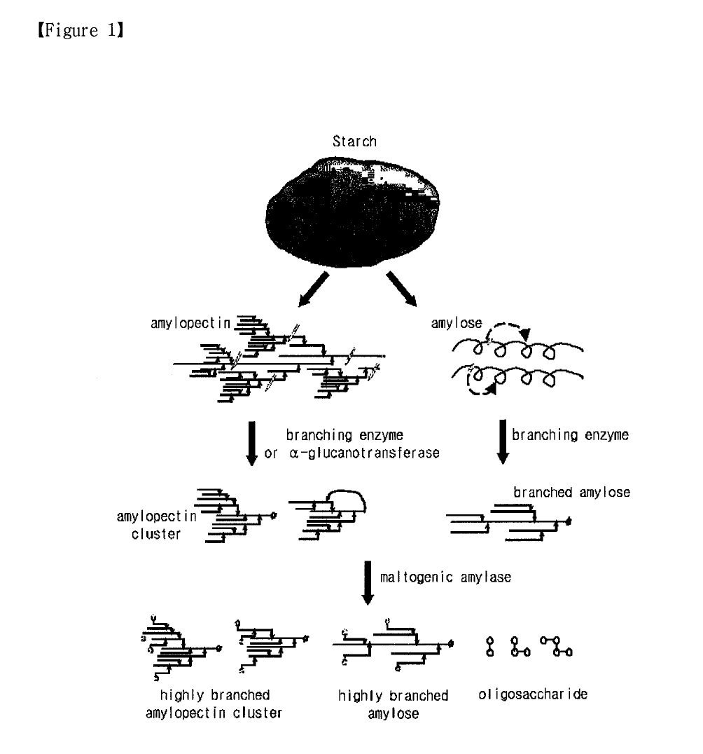

FIG. 2 is a schematic diagram to show the procedure for cloning of a gene encoding branching enzyme of Bacillus subtilis 168.

FIG. 3 an electrophoresis-photogram of branching enzyme of Bacillus subtilis 168 produced by E. coli MC1061.

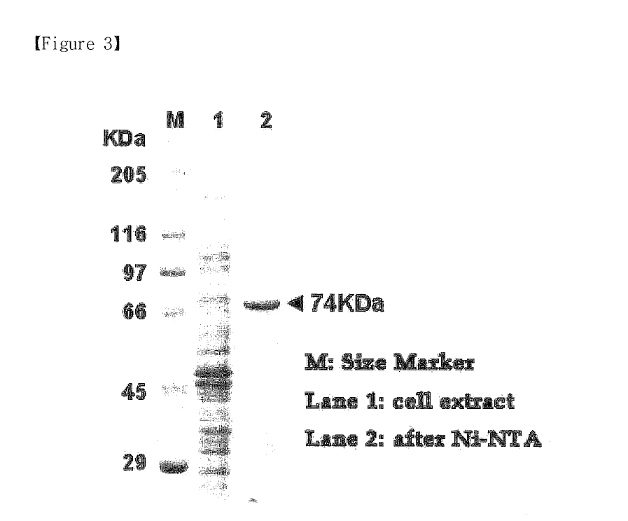

FIG. 4 is a graph to show an activity of recombinant protein, branching enzyme, depending on various pH.

FIG. 5 is a graph to show an activity of recombinant protein, branching enzyme, depending on various temperature.

FIG. 6 is a SEC-MALLS analysis result to show the decrease in molecular weight of amylopectin cluster prepared using α-GTase.

FIG. 7 is a chromatogram to show the side-chain distribution of highly branched amylopectin cluster and highly branched amylopectin cluster.

FIG. 8 is a chromatogram to show the side-chain distribution of branched amylose and highly branched amylose.

FIG. 9 is a TLC chromatogram of branched oligosaccharide which is byproduct produced during the process for preparing the highly branched amylose and amylopectin cluster using BSMA.

### BEST MODE

Hereinafter, the present invention is explained in detail by the following examples. However, the examples are provided for illustration of the present invention, not for limitation thereof.

### Example 1

### Cloning, Production and Characteristics of Branching Enzyme from Bacillus subtilis 168

1-1. Cloning of a Gene Encoding Branching Enzyme

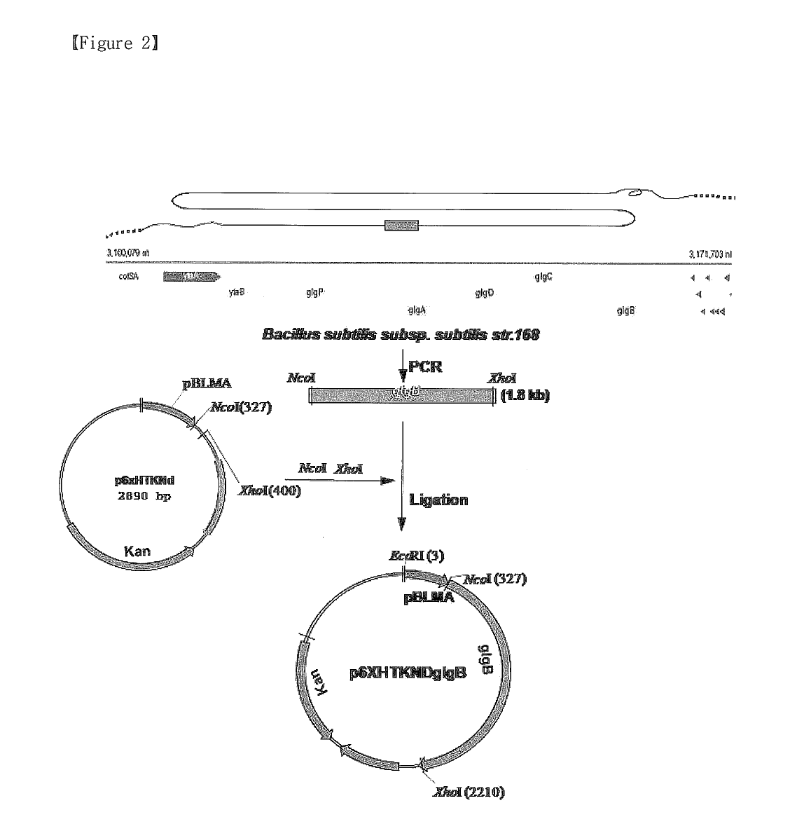

To isolate branching enzyme, the forward primer F-glgB (5′-GAAAGGATGATTCCATGGCCGCTGCCAGC-3′) and the reverse primer R-glgB (5′AAAGAGGAGAGATAAAAAGATGAAAAAACAATGTGTAGCCA-3′) was prepared respectively. And then, PCR was carried out using the mixture of chromosomal DNA of Bacillus subtilis 168 (ATCC 23857D-5), Ex taq polymerase (Takara Phuzo, Tokyo, Japan), Ex taq buffer and dNTP mixture. For PCR, US/7500 realtime PCR system (Corbett company) was used. The reaction was carried out as follows: one time at 95° C. for 5 min as first step, and thirty times repetition at 94° C. for 30 sec and at 55° C. for 30 sec and at 72° C. for 3 min as second step, finally at 72° C. for 7 min. The reaction was terminated by cooling at 4° C. for 4 min. As a result of the above reaction, 1.8 kb of PCR product was obtained. The PCR product was treated with restriction enzymes, NcoI and XhoI, and ligated with the corresponding expression vector p6xHTKNd, preparing p6xHTKNDglgB. The cleavage map of p6xHTKNDglgB was shown in FIG. 2. The p6xHTKNDglgB vector has a gene encoding branching enzyme and has an additional six-histidine tag in 3′-end of the gene of branching enzyme.

For transformation, E. coli MC1061 (New England Biolab inc.) was precultured in 5 mL of LB medium at 37° C. for 12 hr. One mL of the preculture solution was transferred into 50 mL of fresh LB medium, and cultured until the O.D at 600 nm became 0.5. Then, 1.5 mL of the culture medium was centrifuged (7,000×g, 5 min) at 4° C. and then the precipitate was collected and re-suspended with 0.75 mL of transforming solution (50 mL CaCl₂), and kept in ice for 30 min. 15 mL of the suspended solution was mixed with 500 ng of the ligated solution containing p6xHTKNDglgB, and kept in ice for 1 hr, and were heat-shocked at 42° C. for 2 min. After the heat-shocked solution was supplemented by 0.8 mL of LB medium and cultured at 37° C. for 1 hr, it was spreaded on LB agar medium containing kanamycin (final conc. 100 mg/mL) for screening resistant microorganism.

The screened microorganism was inoculated in 5 mL of LB liquid medium containing kanamycin, and cultured at 37° C. for 12 hr and centrifuged to collect microorganism. Plasmid was isolated from the collected microorganism using plasmid isolation kit, and the plasmid was cleaved with restriction enzymes, NcoI and XhoI, which showed the plasmid was containing about 1.8 kb of branching enzyme.

1-2. Preparation of Transformants

The above prepared p6xHTKNDglgB vector was transformed into E. coli MC1061 for screening kanamycin-resistant microorganisms. The screened transformants were inoculated in 3 L of LB liquid medium containing ampicillin, and cultured at 37° C. for 16 hr.

1-3. Purification

The above cultured transformants were collected by centrifugation (4° C., 7,000×g, for 30 min) and resuspended 50 mM of Tris-HCl buffer solution (pH 7.5) containing 300 mM of NaCl and 10 mM of imidazole, and were sonicated. The sonicated solution was centrifuged to obtain supernatant, and the supernatant was heat-treated at 70° C. for 20 min. After heat-treatment, the solution was centrifuged (10,000×g, 30 min) to collect supernatant. From the supernatant, branching enzyme solution was purified using Ni-NTA affinity-chromatography.

1-4. Activity and pH Characteristic of the Enzyme

The enzyme solution (25 μL) in 50 mM of Tris-HCl buffer solution and 0.1% amylose solution in 50 mM of Tris-HCl buffer solution (pH 7.5) were mixed and incubated at 37° C. for 20 min. The reaction was terminated and colored by iodide-HCl solution. The absorbance was measured at 660 nm. One unit of branching enzyme was defined as the amount of enzyme that degraded 1 μg/mL amylose per min compared to amylose standard curve.

To determine the optimum pH of the enzyme, its relative activity was measured using various pH of beta-cyclodextrin.

25 μL of 0.1% (w/v) amylose solution, which is dissolved in 50 mM of sodium acetate buffer solution (pH 4.0˜6.0), 50 mM of sodium phosphate buffer solution (pH 6.0˜8.0) and 50 mM of Tris-HCl of buffer solution (pH 7.0˜10.0) respectively, was added to 25 μL of substrate solution diluted with each buffer solution, and incubated at 30° C. for 20 min to determine the relative activity of the enzyme.

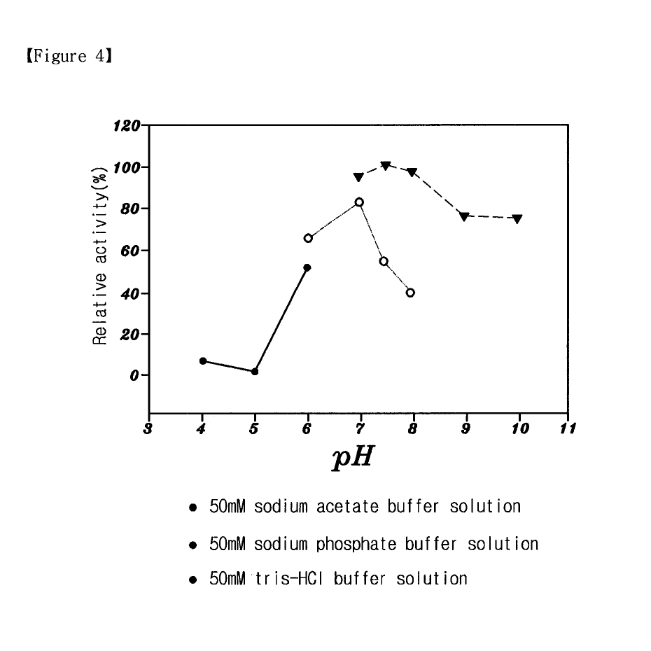

As FIG. 4 shows the activity of recombinant protein, branching enzyme, depending on various pH conditions, the branching enzyme exhibited the maximum activity at pH 7.5, and more than 50% activity at pH 9.0˜10.0.

1-5. Temperature Characteristic of the Enzyme

To determine the optimum temperature of the branching enzyme, 25 μL of the amylose solution in 50 mM Tris-HCl buffer solution (pH 7.5) was heat-treated at various temperature for 5 min. Then, the heat-treated solution was mixed with 25 μL of the branching enzyme, and incubated at each temperature for 20 min to determine its relative activity of enzyme.

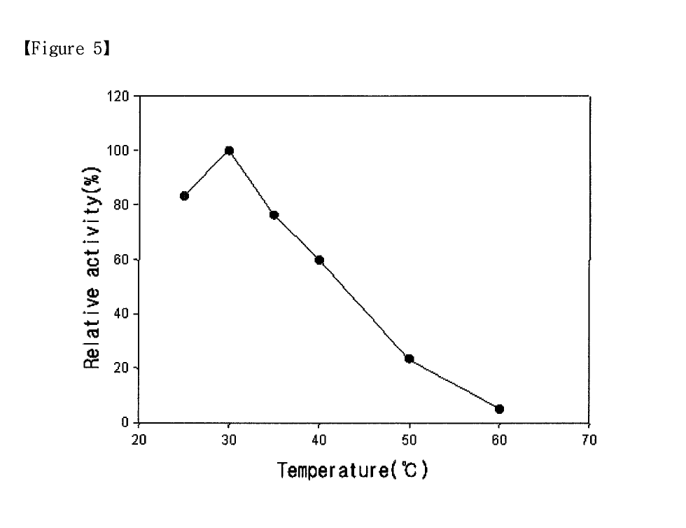

As FIG. 5 shows the activity of recombinant protein, branching enzyme, depending on the temperature, the enzyme exhibited the maximum activity at 30° C.

### MODE FOR INVENTION

### Example 2

### Preparation of Amylopectin Cluster

2-1. Preparation

Waxy rice starch was used as a source of starch.

Alpha-glucanotransferase was prepared by means of that α-glucanotransferase gene of Thermus scotoductus ATCC 27978 was transformed into Bacillus subtilis ISW1214 (Takara Phuzo Corporation) and its expressed enzyme was purified using Ni-affinity chromatography.

5% waxy rice starch solution was prepared using 25 mM phosphate buffer solution (pH 6.5), and was kept in boiling water at 20 min for gelatinization. Then, 100 U/g α-glucanotransferase (100 unit per 1 g of waxy rice starch) was added to the gelatinized starch solution, and its mixture was incubated at 75° C. for 30 min. The reaction was stopped by boiling the mixture for 30 min.

2-2. Measurement of Molecular Weight Using SEC-MALLS (Size Exclusion Chromatography-Multi Angle Laser Light Scattering)

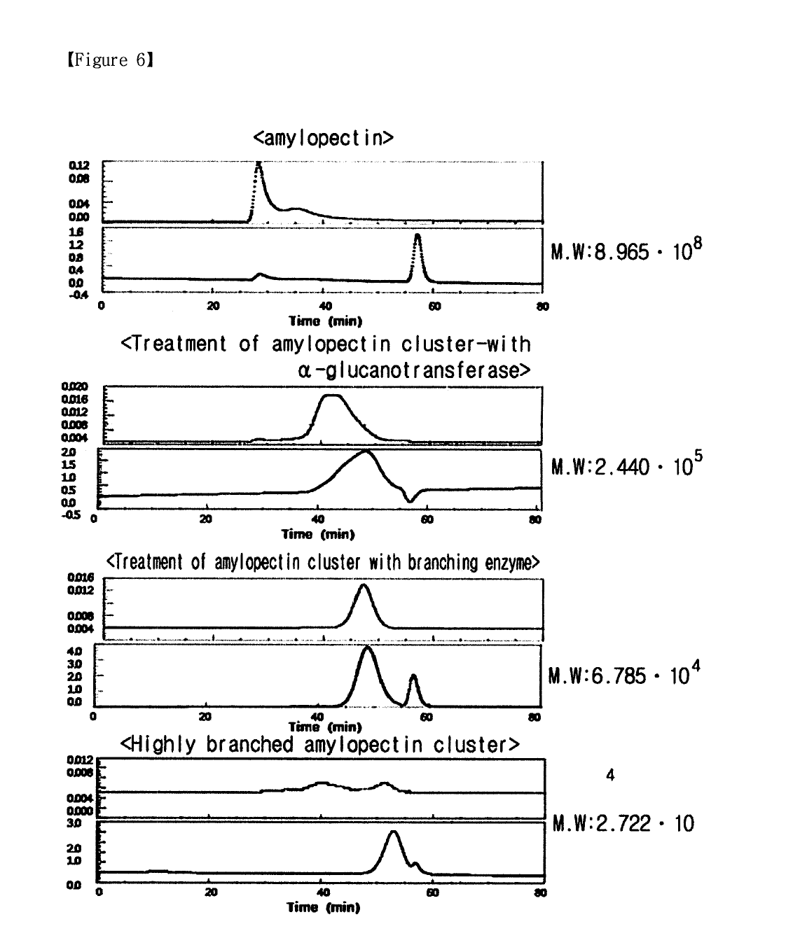

0.5% (5 mg/mL) waxy rice starch and 0.5% (5 mg/mL) amylopectin cluster were boiled for more than 1 hour, and injected into SEC-MALLS for a measurement of their molecular weight respectively. The measurement was carried out using MALLS system manufactured by Wyatt Technology and using column consisted of SUGAR KS-806 (8.0 mmID×300 mm) and SUGAR KS-804 (8.0 mmID×300 mm) connected, manufactured by Shodex company. The result was shown in FIG. 6.

### Example 3

### Preparation of Highly Branched Amylopectin Cluster from Amylopectin Cluster

3-1. Preparation of Highly Branched Amylopectin Cluster

Maltogenic amylase was prepared by means of that maltogenic amylase gene of Bacillus stearothermophilus KCTC 0114BP was transformed into Bacillus subtilis LK87 (graduate school of Korea university, Improvement of the production of foreign proteins using a heterologous secretion vector system in Bacillus subtilis: effects of resistance to glucose-mediated catabolite repression. Mol. cells. 1997 Dec. 31; 7 (6):788˜94.) and its expressed enzyme was purified using Ni-affinity chromatography.

Then, 100 U/g maltogenic amylase (100 unit per 1 g of amylopectin cluster) and the reaction solution of which its reaction is stopped in Example 1 were incubated at 55° C. for 4 hr. The reaction was terminated by boiling for more than 30 min, and the solution was centrifuged at 12,000 rpm for 20 min to remove denatured protein. To remove the salt remaining in the reaction solution, the reaction solution was passed through anion-exchange resin (C100FL, Prolite corporation) and anion exchange resin (A400, Prolite corporation). Then, in order to remove remaining polysaccharides, two volumes of ethanol was added, resulting in precipitation of high molecule of saccharides, and the solution was re-centrifuged, and the supernatant was discarded and the precipitate was freeze-dried, powdered.

3-2. Analysis of the Reaction Solution Using High Performance Ion Exchange Chromatography

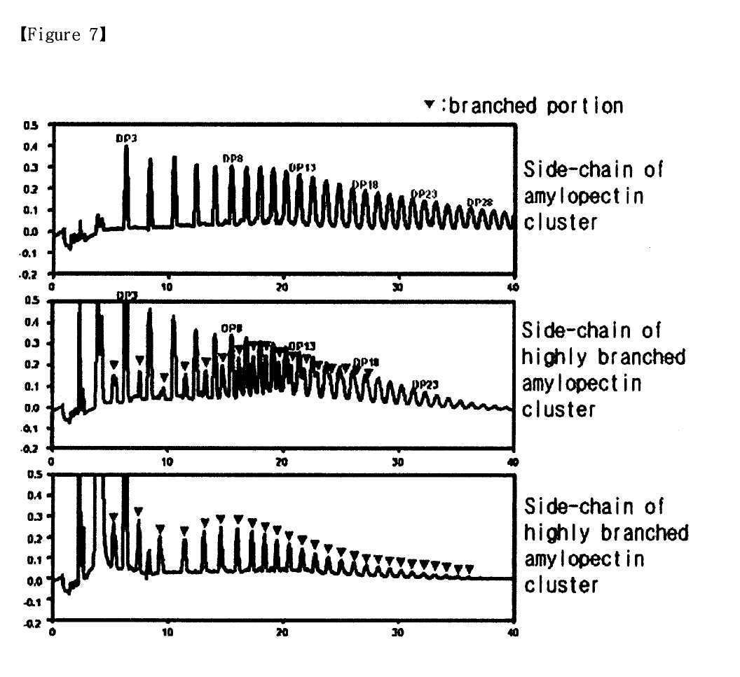

To confirm the distributions and compositions of side-chain in amylopectin cluster and highly branched amylopectin cluster, the above clusters were dissolved in 25 mM of sodium acetate buffer solution (pH 4.3) respectively to prepare 1% solution. The solution was treated with 0.5 U/mg (0.5 unit per 1 mg of substrate) of isoamylase at 60° C. for 48 hr. Each reactant, in which α-1,6 linkage was cleaved, was analysed using high performance ion exchange chromatography (GP40 gradient pump, Dionex corporation) with CaroboPAC™ PA1 (4×50 mm) column. As it is difficult to differentiate branched part in highly branched amylopectin cluster with more than DP 13, was treated with beta-amylase for easiness of analysis. (FIG. 7)

### Example 4

### Hydrolysis Kinetics of Highly Branched Amylopectin Cluster Using Glucoamylase

The Hydrolysis kinetics of amylopectin cluster and highly branched amylopectin cluster were investigated using glucoamylase (Fluka Biochemica.) isolated from Aspergillus niger.

50 μL of various concentrations of the substrate solutions in 50 mM sodium acetate buffer (pH 4.5) were prewarmed at 50° C. for 5 min. 50 μL (0.3 U) of glucoamylase was then added to the substrate solution, and aliquots (20 μL) of the reaction mixture at 60° C. were collected every 30 sec or 1 min for 5 min. The reaction was stopped by addition of 20 μL of 0.1N NaOH. The amount of hydrolysis of amylopectin cluster or highly branched amylopectin cluster was measured using GOD-POD method (glucose determination reagent, Asan pharmaceutical.)

Table 1. shows the measurement and quantification results of the hydrolysis kinetics of amylopectin cluster and highly branched amylopectin cluster by glucoamylase using GOD-POD method.

TABLE 1

| | Kₘ[mg/mL] | K_{cat}[s⁻¹] | K_{cat}/Kₘ[s⁻¹(mg/mL)⁻¹] |
|---|---|---|---|
| amylopectin cluster | 1.14(±0.3) | 52.4(±3.8) | 45.9(±3.34) |
| highly branched | 4.15(±0.2) | 45.9(±2.4) | 11.1(±1.1) |
| amylopectin cluster | | | |

### Example 5

### Preparation of Branched Amylose and Highly Branched Amylose

5-1. Preparation of Branched Amylose

To produce branched amylose, 10 mL of 0.2% amylose (type III extracted from potato, Sigma) in 90% DMSO (dimethyl sulfoxide) was used. To the amylose solution, 10 mL of 200 mM Tris-HCl buffer (pH 7.5) and 50 U/mg of branching enzyme (50 U per 1 mg of substrate) were added with shaking. Then, distilled water was added in order to make 40 mL by final volume. The reaction mixture was prewarmed at 30° C. for 4 hr and the reaction was stopped by heating in boiling water for 10 min. To remove the insoluble salts, two volumes of 100% ethanol was added, and kept at −20° C. for 30 min, and centrifuged at 12,000 rpm, 4° C. for 20 min. After centrifugation, the supernatant was discarded, and the precipitate which contains branched amylose was collected.

5-2. Preparation of Highly Branched Amylose

To produce highly branched amylose, the reactant of Example 5-1 was mixed and resuspended in 50 mM sodium citrate buffer (pH 6.5), and 0.5 U/mg of BSMA (0.5 unit per 1 mg of substrate) was added. The reaction mixture was incubated at 50° C. for 12 hr, and its reaction was terminated by heating in boiling water at 100° C. for 10 min. To remove the produced oligosaccharide, two volumes of 100% ethanol was added, and the solution was kept at −20° C. for 30 min and centrifuged at 12,000 rpm, 4° C. for 20 min, generating the precipitate, highly branched amylose, the supernatant was discarded,

5-3. Side-Chain Analysis of Branched Amylose and Highly Branched Amylose

To analyze the side-chain distributions and compositions of branched amylose and highly branched amylose, the samples were prepared by suspending in 25 mM sodium acetate buffer (pH 4.3) to make 1% solutions. The solutions were incubated with 0.5 U/mg isoamylase (0.5 unit per 1 mg of substrate) at 60° C. for 48 hr. The debranched (α-1,6 linkage was cleaved) samples were analyzed using high performance ion exchange chromatography (GP40 gradient pump, Dionex corporation), coupled with CaroPAV™ PA1 (4×50 mm) column.

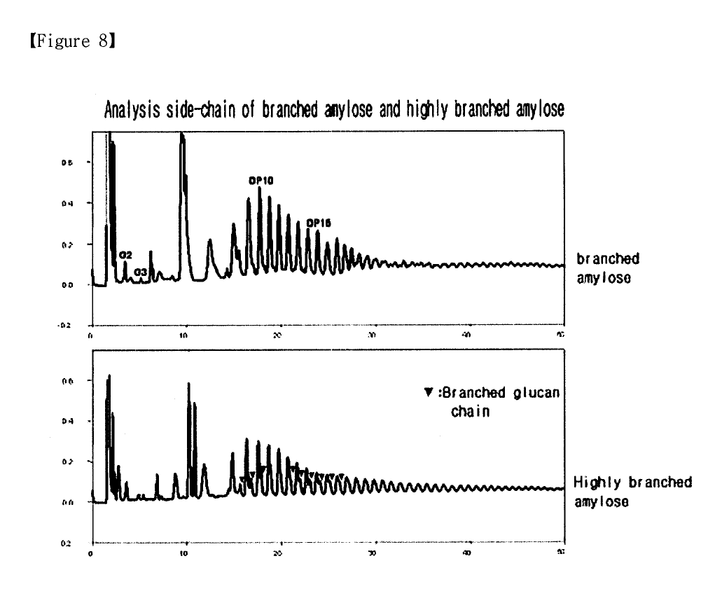

FIG. 8 reveals that highly branched amylose has newly branched parts compared to branched amylose.

### Example 6

### Analysis of Oligosaccharides in Highly Branched Amylopectin Cluster Reaction Solution

To analyze oligosaccharide which is a byproduct in the production of highly branched amylopectin cluster, the samples were collected at various times such as 0.5, 1, 3, 5, 15 hr during the reaction between maltogenic amylase and amylopectin cluster. The samples were mixed with two volumes of ethanol and kept at −20° C. for 30 min, the giant molecules were precipitated, the supernatants were obtained by centrifugation at 12,000 rpm, 4° C. for 20 min. The each supernatant was analyzed using Thin Layer Chromatography Analysis. 1 μL of the samples were spotted on the plate (Whatman K5F silica gel TLC plate) and developed once in a TLC chamber containing a solvent mixture of isopropyl alcohol/ethyl acetate/water (3:1:1 v/v/v). The plate was dried thoroughly and developed by dipping it rapidly into a methanol solution containing 0.3% (w/v) N-(1-naphthyl)-ethylenediamine and 5% (w/v) H₂SO₄. The plate was dried and placed in an oven at 110° C. for 10, min until black spots appeared on the white background.

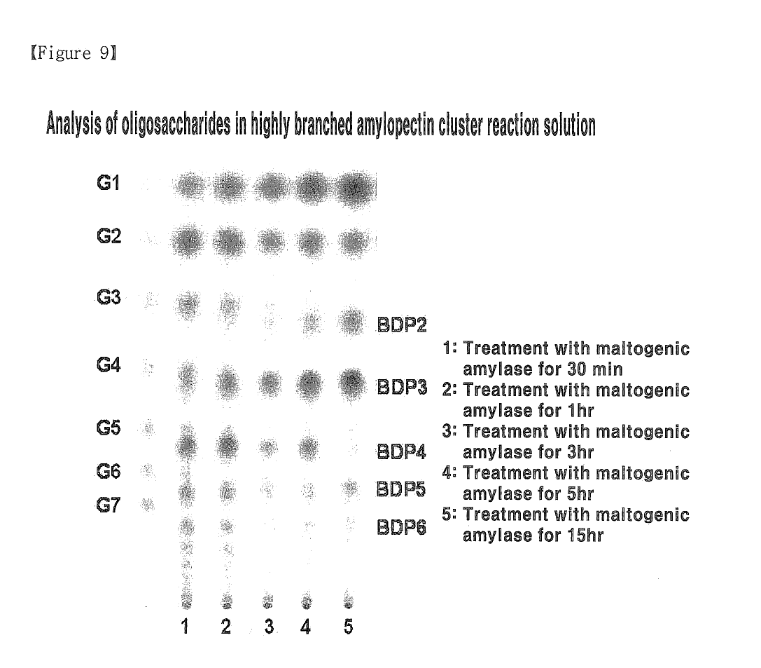

As shown in FIG. 9, long maltooligosacchrides were proven to be converted into 2˜5 BDP of branched oligosaccharides as the reaction time goes.

1. A method for preparing highly branched amylose, highly branched amylopectin cluster or branched oligosaccharide by incubating starch with branching enzyme or alpha-glucanotransferase and maltogenic amylase.

2. The method according to claim 1, which comprises the steps of: incubating starch with branching enzyme isolated from Bacillus subtilis or alpha-glucanotransferase isolated from Thermus scotoductus at 30˜75° C. for 30 min to 4 hr, to produce amylopectin cluster or branched amylose; and treating the obtained amylopectin cluster or branched amylose with maltogenic amylase derived from Bacillus stearothermophilus at 45˜65° C. for 2˜4 hr, to prepare highly branched amylose, highly branched amylopectin cluster or branched oligosaccharide.

3. The method according to claim 1, wherein the starch is selected from the group consisting of starch, grains with starch, amylopectin and amylose.

4. The method according to claim 1, wherein the highly branched amylopectin cluster, has a molecular weight of 10⁴-10⁵ a DP of 6-23 and an alpha-1,6 linkage.

5. The method according to claim 1, wherein the highly branched amylose, has a DP of 8-18 and alpha-1,6 and alpha 1,4 linkages.

6. The method according to claim 1, wherein the branched oligosaccharide, has a 2-5 BDP by TLC.

7. A processed health care food according to claim 1 containing highly branched amylose or highly branched amylopectin cluster.

8. The processed health care food according to claim 7, wherein the food has an effect of anti-diabetes, anti-obesity or continuous energy supply.


SEQUENCE LISTING (2 sequences)

SEQ ID NO: 1 - DNA, 29 bp, Unknown
  PCR Primer Sequence
gaaaggatgattccatggccgctgccagc

SEQ ID NO: 2 - DNA, 41 bp, Unknown
  PCR Primer Sequence
aaagaggagagataaaaagatgaaaaaacaatgtgtagcca